(19) United States
(12) Patent Application Publication
(10) Pub. No.: US 2008/0063689 A1
(43) Pub. Date: Mar. 13, 2008

(54) Dietary supplements containing anthocyanins and methods for using same to enhance creatine absorption and vasodilation

(76) Inventor: Mike Farber, Nutley, NJ (US)

Correspondence Address: Reine H. Glanz;Suite 6C, 245 E 58th, New York, NY, 10022, US

(21) Appl. No.: 11/519,656

(22) Filed: Sep. 12, 2006

(51) Int. Cl. A61K 31/7048; A61K 31/7076; A61K 31/70; A61K 31/7012; A61K 31/385; A61K 31/198; A61K 31/22; A61K 31/185; A61K 31/205; A61K 36/258

(52) U.S. Cl. 424/439; 514/27; 514/456; 514/554; 514/546; 514/23; 514/53; 514/58; 514/47; 514/561; 514/553; 514/565; 424/728; 424/725; 424/756; 424/617; 514/440

(57) ABSTRACT

A problem with optimizing the effects of creatine has been the need to utilize large amounts of high glycemic carbohydrates to spike insulin in order to augment the flow of creatine (and other nutrients) into the muscle cell. Based upon current research, an insulin response strong enough to ensure absorption is possible without the need for dramatic carbohydrate load. Utilizing anthocyanins, such as cyanidin-3-glycoside and other potentiators, creatine can be absorbed by the muscles without overloading the system with excess calories from glucose (carbohydrates). U.S. Patent Application Publication No. 20040224035, a considerably lower daily intake of carbohydrates is necessary to achieve creatine absorption. The reduced caloric value necessary to produce the equivalent creatine absorption translates to lower levels of glucose needed for clearance and could have substantial; effects on body fat storage or on body composition over an extended period of time since the caloric intake is reduced by almost 1200 calories over the method cited.

### BACKGROUND OF THE INVENTION

Creatine has long been known to increase energy and endurance. During WWI German soldiers were given rations of creatine to take before battle. A problem with optimizing the effects of creatine has been the need to utilize large amounts of high glycemic carbohydrates to spike insulin in order to augment the flow of creatine (and other nutrients) into the muscle cell.

Based upon current research, an insulin response strong enough to ensure absorption is possible without the need for dramatic carbohydrate load. Utilizing anthocyanins, such as cyanidin-3-glycoside and other potentiators insulin levels can be obtained that result in creatine absorption without overloading the system with excess calories from glucose (carbohydrates). Materials derived from elderberry, cherry, purple corn, black soybean and other natural sources of anthocyanins can be administered orally to humans and or animals for the purposes of controlling blood glucose as well as improving glucose tolerance. In the case where insulin functioning is impaired, such as Type 11 diabetes, controlling glucose metabolism is essential. Anthocyanins have also been recently shown to have an effect on endothelial nitric oxide production and on vasodilation. Thus, administration of anthocyanins can also be used for the purpose of enhancing the transport of nutrients into tissues such as skeletal muscle for the goals of athletic performance, control of bodyweight and or body fat levels. Similarly related, such administration can also be used for the purpose of enhancing creatine transport into excitable tissues such as skeletal muscle.

U.S. Patent Application Publication No. 20040224035 discloses the use of cinnamon extract to increase the absorption of creatine. However, a high amount of carbohydrates needs to be consumed in order for this process to occur.

### SUMMARY

Disclosed herein is a dietary supplement comprising an anthocyanin extract containing cyanidin 3 glycoside, delphinium 3 glycoside or palerginium 3 galactoside or mixtures thereof and a nutrient, or a derivative or a precursor thereof without or with a carbohydrate and a method of increasing the absorption of nutrients into mammalian tissue, therefore enhancing nutrient transport, and athletic performance comprising administration of the dietary supplement.

Accordingly, it is an object of this invention to provide a method and a dietary supplement which will enhance the absorption of nutrients into skeletal muscles. More specifically it is the object of the invention to provide a method and a dietary supplement that will enhance the absorption of creatine into skeletal muscle. It is further an object of the invention to stimulate an insulin dependant pathway for absorption of creatine into the skeletal muscle.

Also disclosed herein a dietary supplement comprising anthocyanin extracts specifically containing cyanidin-3-glycoside, dlephionium 3 glycosides and pelarginum 3 or a mixture or derivatives thereof and a method of losing weight and reducing body fat comprising administration of these dietary supplement.

Accordingly it is an object of this invention to provide a method and a dietary supplement which will promote weight loss and body fat reduction.

### FIELD OF THE INVENTION

The present invention is directed to dietary supplements containing anthocyanins, specifically cyaniding-3-glycosides and/or delphinium-3-glycosides, and to methods of using these dietary supplements to enhance the transport of nutrients and to promote athletic performance and weight loss both in humans and animals.

Type 11 diabetes is quickly becoming an epidemic within the United States and is on the rise in various other countries. This increased incidence has been related to the diets containing high fat intake and repeated ingestion of refined foods and sugars, coupled with low fiber and vegetable intake. Diet along with aging causes a deterioration in the manner in which the body metabolizes blood glucose. When the body can no longer properly metabolize glucose there is a tendency to store glucose as body fat occurs. Diabetes is also connected to various other ailments such as heart disease, hypertension, obesity, retinopathy, etc. There is a known link between insulin resistance and visceral adiposity.

It has long been known that natural and/or synthetic substances can aid in the control of blood glucose and enhance nutrient transport. Such substances are utilized by the body through a variety of mechanisms. For example some substances act through the mimicking of the effects of exogenous insulin and are therefore capable of replacing exogenous insulin. Synthetic insulin is one such substance. Other naturally occurring substances include the amino acid taurine, 4-hydroxyisoleucine, arginine and vanadium. Although these substances act to imitate insulin, they have yet to be developed into viable alternatives of treatments for disorders of glucose metabolism.

Still other substances act directly to increase what is known as insulin sensitivity or lower glucose tolerance. High glucose tolerance forces the body to generate additional insulin in an effort to lower blood glucose levels. This causes repeated stress on the pancreatic beta cells and is thought to be a key factor in the development of Type 11 diabetes. Substances that lower glucose tolerance include alpha lipoic acid, d-pinitol and myo-inositol. These substances work alongside endogenous insulin at the receptor level to increase insulin sensitivity or lower glucose tolerance.

In the past chromium was thought to aid in weight loss by controlling blood glucose levels and affecting fatty acid deposits. However, its effects were greatly limited. Cinnamon, a rich source of chromium, has also been used to control glucose and its effects were at one time thought to be related to chromium. It has lately been demonstrated that certain water soluble compounds in cinnamon are strong potentiators of insulin (J Agricultural Food Chem 2000 48:849-852).

A recent study of methylhydroxychlacone polymers (MHCP) compared the effects of MHCPs to that of insulin on adipocytes (Jarrill-Taylor, et al. J. Amer. College Nutr. 2001 20:337-336). The study demonstrated that MHCP stimulated glucose absorption and glycogen synthesis to a similar level as insulin.

Recent studies have shown that bioavailable anthocyanins present in fruit (among other natural substances) stimulate insulin secretion from pancreatic beta cells at glucose concentrations that are within the physiological norms of most individuals. Specifically, the two anthocyanins cyanidin-3-glycoside and delphinium 3-glycoside, demonstrated the highest abilities to stimulate insulin secretion. (J. Agric. Food Chem 2005 Jan. 12; 53:28-31).

Furthermore, the extracts from cherries and other fruits have been shown in recent studies to have anti-diabetic effects and this effect has been correlated to the presence of the glucoside Cyanidin-3-glycoside. In fact, recent filings for patents refer to the utility of these extracts containing cyaniidin-3-glycoside (and other anthocyanins) as being used to control blood glucose levels (U.S. Patent Application Publication Nos. 20050084548, 20060025353 and 20060025354).

It is also been noted that the chemical structure of the anthocyanins in question have similar chemical structure in their functional groups to that of the above referenced cinnamon extract methylhydroxychalcone polymer.

In broad terms nutrient transport involves the deposit of various nutrients from aminos to minerals into various tissues. For example, the insulin cascade triggers the Glut 4 transport system that is responsible for nutrients such as amino acids and creatine entering into skeletal muscle cells. Typically water is driven into the cells at the same time.

Creatine is a natural dietary product primarily found in animal tissue and is mainly stored in the skeletal muscle tissues. The storage of creatine occurs predominantly in its phosphorylated form but occurs to a lesser extent in its free form. The level of creatine normally found in mammalian skeletal muscle can be increased by creatine supplementation.

The energy for all muscular work in the body is adenosine triphosphate (ATP). During intense exercise ATP is rapidly consumed by the muscles and is converted to adenosine diphosphate (ADP), a lower energy form. However, ATP is not stored in large quantities in the body, therefore, to enable the muscles in the body to work for a prolonged period of time, ATP must be effectively and quickly replenished. This is accomplished most effectively through the addition of phosphorylated creatine. Thus, the primary function of phosphorylated creatine is to act as a catalyst to increase the supply of the phosphate needed to restore ATP

Once creatine has been broken down to create ADP, the creatine is absorbed into muscle tissue by an active transport system that typically involves an insulin dependant pathway (GLUT 4). Studies by Stengee, et al. showed that creatine absorption could be stimulated by sufficiently high insulin levels, supraphysilogical. Also, studies by Green, et al. showed that creatine absorption was stimulated by high glucose concentrations that caused insulin spiking but that the glucose level to be effective approached the limits of palatability.

Thus, there still exists a need for a viable method to increase the absorption of creatine (and other nutrients) into mammalian tissue such as skeletal muscle without the requirement to supplement carbohydrates or to minimally supplement with carbohydrates below normal physiological parameters.

What is claimed:

1. A dietary supplement comprising: an extract rich in anthocyanins, and creatine.

2. The dietary supplement of claim 1 wherein said anthocyanins comprises one of the following anthocyanins cyanidin-3-glycoside, delphinium-3-glycoside or a mixture thereof.

3. A dietary supplement of claim 1 wherein said creatine comprises one of following: creatine monohydrate, dicreatine malate, creatine citrate, creatine pyruvate, alkaline creatine, magnesium creatine chelate, creatine ethyl ester, tricreatine malate, creatine hydrates, creatine phosphate or other creatine derivatives.

4. A dietary supplement of claim 1 wherein said creatine is selected from group consisting of glyco-cyamines, guanidinopropionic acids, trimethyl glycine, and s-adenosyl methionines.

5. The dietary supplement of claim 1 further comprising at least one carbohydrate in the amount of 1 mg to 950 mg per gram of the dietary supplement.

6. The dietary supplement of claim 5 wherein said carbohydrate is selected from the group of consisting of dextrose, glucose, maltodextrin, maltose and trehalose,

7. The dietary supplement of claim 5 wherein said carbohydrate is selected from the group consisting of complex carbohydrates such as potato starch.

8. The dietary supplement of claim 1 further comprising free amino acids.

9. The dietary supplement of claim 8 wherein said free amino acids includes taurine, glutamine, carnitine, citrulline, leucine, glycine, arginine, alanine, and the derivatives thereof

10. The dietary supplement of claim 1 further comprising a blood glucose-modifying or insulin modifying agent.

11. The dietary supplement of claim 10 wherein said blood glucose modifying or blood insulin modifying agent is selected from the group consisting of vanadium, chromium, banaba extract, corosolic acid, alpha lipoic acid, d-pinitol, panax ginseng, ginseng extract, ginger extract, cinnamon, cinnamon extract, methylhydroxychalcone polymers and 4-hydroxyisoleucine.

12. The dietary supplement of claim 1 further comprising vitamins, vitamin derivatives, minerals, vasodilation agents, vitamin cofactors, herbs and herbal extracts.

13. The dietary supplement of claim 1 further comprising a lactate buffering agent.

14. The dietary supplement of claim 1 wherein the dietary supplement is in the form of a powder that can be mixed with a fluid to form a beverage.

15. The dietary supplement of claim 14 wherein the fluid is water.

16. The dietary supplement of claim 2 wherein said anthocyanins comprise 0.1 mg to 1000 mg and said cyanidin-3-glycoside or delphinium-3-glycoside comprises 0.1 mg to 600 MG per gram of the dietary supplement.

17. The dietary supplement of claim 1 where element said creatine comprises 1 mg to 950 mg per gram of the dietary supplement.

18. The dietary supplement of claim 1 wherein administration of the dietary supplement in an amount of 200 mg to 500 grams daily at various intervals for a period of 4 to 30 days creates an increase in total creatine content of skeletal muscle.

19. A method of increasing the absorption of creatine into skeletal muscle comprising the step of ingesting a dietary supplement comprising anthocyanins and creatine.

20. A method of enhancing athletic performance comprising the step ingesting a dietary supplement comprising anthocyanins and creatine.
